(19) United States
(12) Patent Application Publication
(10) Pub. No.: US 2024/0269381 A1
(43) Pub. Date: Aug. 15, 2024

(54) CHAIN COMPOSITION PREPARATION APPARATUS AND PREPARATION METHOD

(71) Applicant: SUZHOU YIBEN LIFE TECHNOLOGY CO., LTD, Suzhou (CN)

(72) Inventors: Guoxiang SUN, Suzhou (CN); Yonghua DONG, Suzhou (CN)

(73) Assignee: SUZHOU YIBEN LIFE TECHNOLOGY CO., LTD, Suzhou, JS (CN)

(21) Appl. No.: 18/642,734

(22) Filed: Apr. 22, 2024

Related U.S. Application Data
(63) Continuation of application No. PCT/CN2022/126542, filed on Oct. 20, 2022.

(30) Foreign Application Priority Data
Oct. 20, 2021 (CN) 202111219493.4

(51) Int. Cl. A61M 5/168; A61M 5/14; A61M 5/48

(52) U.S. Cl. CPC A61M 5/16881; A61M 5/1407; A61M 5/486

(57) ABSTRACT

Disclosed is a chain composition preparation apparatus, comprising a filling part, an injection part, a first connecting tube, a second connecting tube, a distribution valve and an outlet fluid tube. The filling part is in communication with the injection part via the first connecting tube, the distribution valve is in communication with the first connecting tube via the second connecting tube, and the outlet fluid tube is connected to the distribution valve; the distribution valve is also used to connect to an external gas supply part and a liquid supply part. The filling part is used to suck gas and liquid to sequentially enter the second connecting tube, to form a drug in an alternating gas-liquid dosage form. Thus, integration of drug configuration and drug injection can be achieved, so that convenience and efficiency of drug injection is improved.

### BACKGROUND

### Technical Field

This invention relates to a chain-like composition (also as chain composition) preparation apparatus and preparation method, and belongs to the field of medical device technology.

### Related Art

With the advancement of medical technology, a transcatheter arterial infusion therapy (catheter-based arterial infusion chemotherapy) technique has emerged. This method is a crucial intervention for cancer treatment, frequently utilized in areas like the radial artery. This approach involves inserting a microcatheter into the target area through the radial artery, and then administering therapeutic drugs through the microcatheter, thus achieving precise infusion treatment while significantly minimizing side effects.

A crucial aspect of transcatheter arterial infusion therapy involves controlling the pressure and speed of drug infusion. If the pressure or speed is too high, there's a risk of drug reflux or the microcatheter being expelled from the artery. Conversely, if it's too low, the drug cannot effectively reach the tumor's interior. Operators are required to manually adjust the pressure and speed through repeated imaging, then rely on their memory to continue the drug infusion. This approach brings subjective biases and thus fails to provide optimal and consistent performance.

Therefore, there is a need for the formulation and injection system of a chain composition, to create a chain composition formed by alternating different media. For the structure of the chain composition, one can refer to Chinese patent application No. 201880021772.X, filed by the applicant on Mar. 26, 2018.

### SUMMARY

To solve the problems in the related art, the present disclosure chiefly provides a chain composition preparation apparatus.

The present disclosure further provides a chain composition preparation method.

To achieve the object, the present disclosure provides the following technical solution:

According to a first aspect of embodiments of the present disclosure, provided is a chain composition preparation apparatus, comprising:

a distribution valve, comprising multiple distribution ports;

a filling part, connected to one of the multiple distribution ports through a first connecting tube, allowing gas or liquid to enter the first connecting tube from the distribution valve, to prepare a chain composition of alternated media;

an injection part, connected to another one of the multiple distribution ports through the first connecting tube, for delivering the chain composition within the first connecting tube;

an outlet fluid tube, connected to other one of the multiple distribution ports to deliver the chain composition from the distribution valve.

Preferably, the filling part comprises a first syringe and a first connecting valve,

the first connecting valve at least has a first valve outlet, a second valve outlet and a third valve outlet, which are connectable with each other,

the first syringe being connected to the first valve outlet, and the third valve outlet being used for evacuation.

Preferably, the injection section comprises a second syringe and a second connecting valve,

the second connecting valve at least has a fourth valve outlet, a fifth valve outlet, and a sixth valve outlet which are one-to-one connectable,

the second syringe is connected to the fourth valve outlet,

the fifth valve outlet is connected to the second valve outlet through the first connecting tube,

the sixth valve outlet is used to connect with a saline supply device.

Preferably, the distribution valve has a common connecting end,

the distribution valve further has a plurality of distribution ports in outer wall thereof,

the common connecting end is rotatable around a central axis of the distribution valve to communicate with any one of the distribution ports;

the second connecting tube is connected to the first connecting tube at a proximal end, while the second connecting tube is connected to the common connecting port at a distal end,

one of the multiple distribution ports is connected to the outlet fluid tube, while the remaining distribution ports is capable to connect to different gas supply parts or liquid supply parts.

Preferably, the chain composition preparation apparatus further comprises a pressure sensor, wherein the pressure sensor is installed on the first connecting tube between the fifth valve outlet and a proximal end of the second connecting tube.

Preferably, both the first connecting tube and the second connecting tube are made of Teflon.

Preferably, the filling part further includes a syringe drive unit,

the first syringe comprises a first syringe tube and a first syringe rod,

the first syringe tube is connected to the first valve outlet at a distal end thereof, and the first syringe rod is movably inserted inside the first syringe tube,

the syringe drive unit is connected to the first syringe rod, to drive the first syringe rod to reciprocate inside the first syringe tube.

Preferably, the filling part further comprises a valve driving unit, and the first connecting valve comprises a first valve body and a first valve core,

the first valve body has a central hole extending axially, and an outer wall of the first valve body is provided with the first valve outlet, the second valve outlet, and the third valve outlet, all of which extend radially and are connected to the central hole;

the first valve core has a curved passage that runs through its outer wall,

the first valve core is rotatably placed inside the central hole and is connected to the valve driving unit,

the first valve core is capable to rotate around the central axis of the first valve body, allowing both ends of the curved passage to communicate with any two of the first valve outlet, the second valve outlet, and the third valve outlet.

According to a third aspect of the embodiments of the present disclosure, provided is a chain composition preparation method using the chain composition preparation apparatus.

Preferably, using the distribution valve to formulate a chain composition with alternating different media,

the chain composition comprises at least three different media.

According to a second aspect of the embodiments of the present disclosure, provided is a chain composition preparation apparatus, comprising:

a distribution valve, comprising multiple interconnected distribution ports;

an outlet fluid tube, connected to one of the distribution ports of the distribution valve; and

a plurality of filling parts, each connected to one of distribution port of the distribution valve other from the one connected to the outlet fluid tube, and each connected to a gas supply part or a liquid supply part, to allow gases or liquids to flow through the distribution valve into the outlet fluid tube to prepare a chain composition with different media alternating.

Preferably, the filling parts are configured to reciprocate and aspirate gas or liquid from the gas supply parts or liquid supply parts and deliver the gas or liquid into the outlet fluid tube,

the filling parts are further configured to aspirate gas from the outlet fluid tube to isolate the liquid from the rest of the liquid.

Preferably, the filling part comprises a first syringe and a first connecting valve,

the first connecting valve at least has a first valve outlet, a second valve outlet and a third valve outlet, which are connectable with each other,

the first syringe being connected to the first valve outlet,

the second valve outlet is connected to the distribution valve through one of its distribution ports,

the third valve outlet is configured to connect with the gas supply part or the liquid supply part.

Preferably, among the plurality of filling parts, only one of them is configured to connect with the gas supply part, while the rest are used to connect with different liquid supply parts.

According to a fourth aspect of the embodiments of the present disclosure, provided is a chain composition preparation method using the chain composition preparation apparatus comprising the following steps:

aspirating a first medium from a liquid supply part through a first filling part, and push it into the outlet fluid tube through the distribution valve,

aspirating gas from a gas supply device through a second filling part, and pushing it into the outlet fluid tube through the distribution valve,

aspirating gas from the outlet fluid tube through the first filling part to isolate the first medium from the rest of the medium,

aspirating a second medium from another liquid supply part through a third filling part, and then pushing it into the outlet fluid tube through the distribution valve;

aspirating gas from the gas supply device through the second filling part and push it into the outlet fluid tube through the distribution valve;

aspirating gas from the outlet fluid tube through the third filling part, to isolate the second medium from the rest of the medium;

aspirating a Nth medium from a Nth liquid supply part through a Nth filling part, and pushing it into the outlet fluid tube through the distribution valve,

repeating above steps until a chain composition of alternating media is prepared;

the Nth medium is different from the first medium or the second medium within the outlet fluid tube.

The invention's chain composition preparation apparatus continuously sucks air from the first and second connecting tubes using the filling part, allowing physiological saline (normal saline) to enter the second connecting tube to evacuate the first and second connecting tubes until they are filled with saline. Subsequently, the apparatus connects with various gas or liquid supply parts through adjustment of the distribution valve, and alternately draws gases and liquids using the filling part to exhaust saline, thereby forming an alternating gas-liquid dosage form (the chain composition) within the second connecting tube. Finally, the apparatus draws physiological saline using the injection part, then pushes it into the first connecting tube, thereby extruding the chain composition from the second connecting tube through the distribution valve into the outlet fluid tube for injection to the patient. This integration of drug preparation and injection introduces increased convenience, efficiency, and precision in preparation and administration, as well as reduced size and cost of the chain composition preparation apparatus. The apparatus enables on-site preparation of drugs, expanding the range of drug preparation applications, achieving a new method of medication delivery where drugs are delivered to the body while being prepared, and precisely delivering drugs to specific locations in arteries or tissues.

### BRIEF DESCRIPTION OF THE DRAWINGS

FIG. 1 is a structural diagram of a chain composition preparation apparatus provided in the first embodiment of the present invention;

FIG. 2 is a schematic diagram of the disassembled structure of the first syringe;

FIG. 3 is a partial sectional view of the first connection valve;

FIG. 4 is a structural diagram of the filling part;

FIG. 5 is a structural diagram of the distribution valve;

FIG. 6 is a structural diagram of a chain composition preparation apparatus provided in the third embodiment of the present invention;

FIG. 7 is a structural diagram of the gas aspirated by the filling part according to the third embodiment of this invention.

### DETAILED DESCRIPTION

The following provides a detailed and specific explanation of the technical content of this invention, incorporating the accompanying figures and specific examples.

### First Embodiment

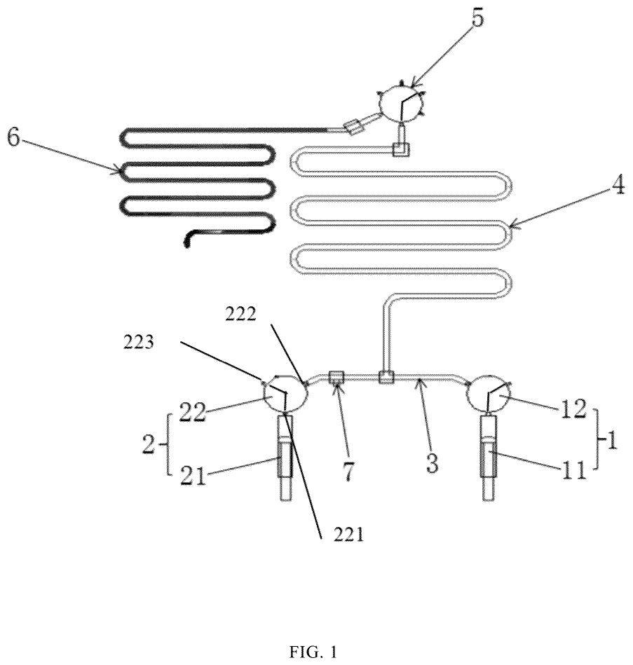

As shown in FIG. 1, the present invention provides a device for preparing a chain composition, which is exemplified in the present embodiment by the preparation and injection systems for alternating gas and liquid substances. The device comprises: a filling part 1, an injection part 2, a first connecting tube 3, a second connecting tube 4, a distribution valve 5, and an outlet fluid tube 6.

The filling part 1 is connected to the injection part 2 through the first connecting tube 3. The distribution valve 5 is connected to the first connecting tube 3 via the second connecting tube 4. The outlet fluid tube 6 is connected to the distribution valve 5. The distribution valve 5 is also used to connect to an external gas supply part (which can alternatively be left unconnected, to be open for air) and a liquid supply part. The filling part 1 is used to aspirate gas from a gas supply device and aspirate liquid from a liquid supply part alternatively into the second connecting tube 4, to form a chain-like composition (chain composition) of alternating media (in this embodiment, an example is provided of a gas-liquid alternating agent formulation). The injection part 2 is used to push the chain composition through the distribution valve 5 into the outlet fluid tube 6.

In practical application, firstly, the air is continuously aspirated from the first connecting tube 3 and the second connecting tube 4 using the filling part 1, allowing physiological saline (normal saline, hereafter “saline” in short) to enter the second connecting tube 4 to evacuate the first connecting tube 3 and the second connecting tube 4, until they are filled with saline. Subsequently, the distribution valve is connected with various gas or liquid supply parts so as to drain the saline by alternately aspirating gases and/or liquids by the filling part 1. Thereby, a chain composition of an alternating gas-liquid formulation is prepared within the second connecting tube 4. Finally, the saline is aspirated by the injection part 2, and then injected into the first connecting tube 3, thereby forcing the chain composition from the second connecting tube 4 through the distribution valve 5 into the outlet fluid tube 6 for injection to the patient. This integrated approach allows for the preparation and injection of drugs using the present chain composition preparation device, offering high efficiency, convenient operation, and precise formulation (the dosage of different media can be precisely controlled). As a result, doctors can prepare drugs on-site, expanding the range of drug preparation applications. Therefore, a new method of medication delivery is achieved, in which the drug (the chain composition) is injected into the human body simultaneously with its on-site preparation, and the drug is precisely delivered to specific locations in arteries or tissues.

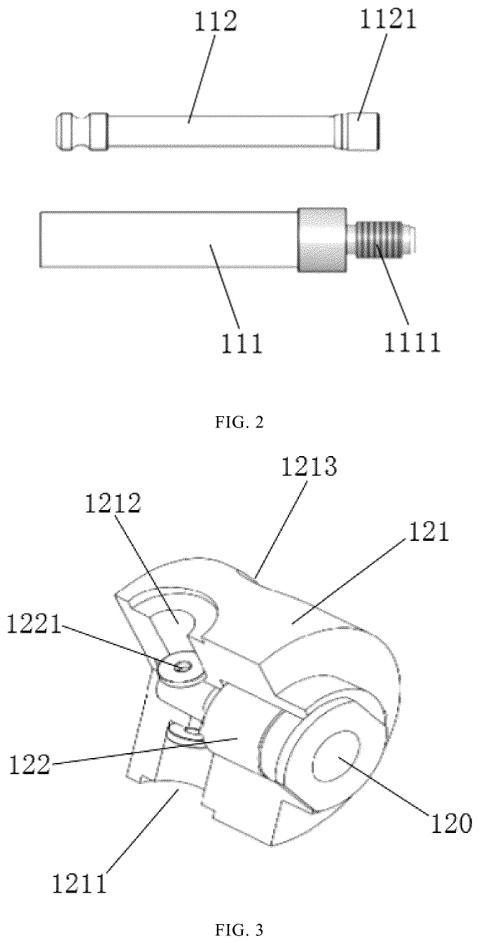

In the present embodiment, specifically, the filling part 1 includes a first syringe 11 and a first connecting valve 12. Referring to FIG. 2, the first syringe 11 comprises a first syringe tube 111 and a first syringe rod 112. The first syringe tube 111 is generally tubular in shape, with a threaded connection part 1111 at its distal end. This threaded connection part 1111 is used to threadably connect with the first connecting valve 12, thereby enhancing the stability of the connection between the first syringe tube 111 and the first connecting valve 12. The first syringe rod 112 slidably extends within the first syringe tube 111, with a head 1121 at its distal end. This head 1121 is made of silicone, rubber or other materials to ensure the sealing between the head 1121 and the first syringe tube 111.

As shown in FIG. 3, the first connecting valve 12 comprises a first valve body 121 and a first valve core 122. In the present embodiment, the first valve body 121 is tubular in shape, with a central hole 120 extending along an axial direction of the first valve body 121. The outer wall of the first valve body 121 is provided with a first valve outlet 1211, a second valve outlet 1212, and a third valve outlet 1213, each of which extends along radical direction of the first valve body 121. The first valve outlet 1211, the second valve outlet 1212, and the third valve outlet 1213 are connected to the central hole 120. The angle between two adjacent valve outlets is 120°. Additionally, the threaded connection part 1111 of the first syringe tube 111 connects the first valve outlet 1211; the second valve outlet 1212 is connected with the first connecting tube 3, and the third valve outlet 1213 is used for evacuation. The first valve core 122 is tubular in shape, with a curved passage 1221 penetrating through the outer wall of the first valve core 122. The curved passage 1221 has a curved angle of 120°. The first valve core 122 is rotatably placed inside a hole of the first valve body 121, to allow the two ends of the curved passage 1221 to connect any two of the first valve outlet 1211, the second valve outlet 1212, and the third valve outlet 1213 (it is shown in FIG. 3 that the curved passage 1221 connects the first valve outlet 1211 and the second valve outlet 1212). When the two ends of the curved passage 1221 are connected to the first valve outlet 1211 and the second valve outlet 1212, the first plug 11 can aspirate or discharge air or saline into the first connecting tube 3. When the two ends of the curved passage 1221 are connected to the first valve outlet 1211 and the third valve outlet 1213, air or saline can be expelled to the outside using the first plug 11 to be ready for next aspiration of the air or saline.

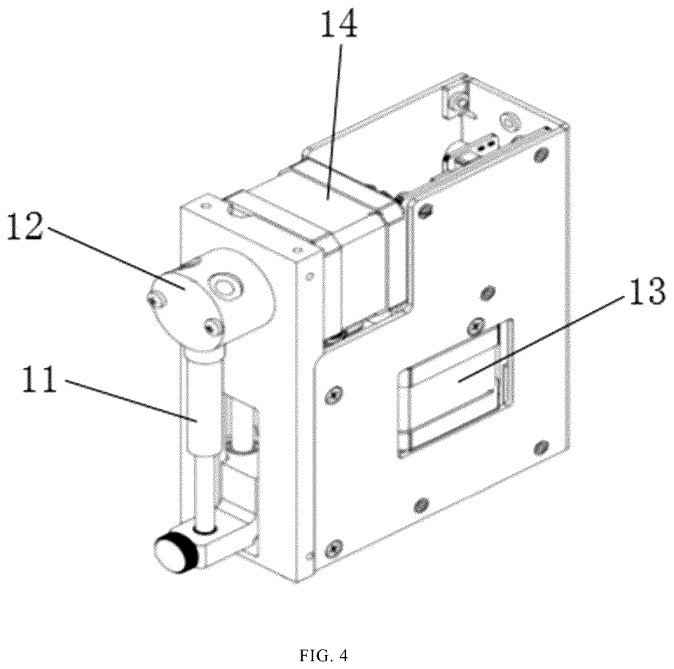

As shown in FIG. 4, in the present embodiment, the filling part 1 further includes a syringe drive part 13. The syringe drive part 13 is connected to the first syringe rod 112, to drive it to reciprocate within the first syringe tube 111. Additionally, the syringe drive part 13 can be connected to a control system, thus allowing for precise control of the movement distance of the first syringe rod 112.

Continuing to refer to FIG. 4, in the present embodiment, the filling part 1 further includes a valve driving unit 14. The valve driving unit 14 is connected to the first valve core 122, to drive the first valve core 122 to rotate around a central axis of the first valve body 121. Therefore, each end of the curved passage 1221 is connected to one of the first valve outlet 1211, the second valve outlet 1212, and the third valve outlet 1213. The valve drive part 14 can also be connected to a control system, allowing precise control of the rotation angle of the first valve core 122 by the control system.

In the present embodiment, the chain composition preparation apparatus also includes a displacement sensor. The displacement sensor is positioned on one side of the first syringe rod 112 to detect the movement distance of the first syringe rod 112 and feed it back to the control system. Additionally, the chain composition preparation apparatus further includes an angle sensor. The angle sensor is positioned on one side of the first valve core 122 to detect the rotation angle of the first valve core 122 and feed it back to the control system.

Similarly, the injection part 2 comprises a second syringe 21 and a second connecting valve 22. The structures of the second syringe 21 and the second connecting valve 22 are similar to those of the first syringe 11 and the first connecting valve 12, which will not be elaborated on here. The difference lies in that the second connecting valve 22 is opened with three outlets: a fourth outlet 221, a fifth outlet 222, and a sixth outlet 223, any two of which can be connected. As is shown in FIG. 1, the second syringe 21 is mounted at the fourth outlet 221, and the sixth outlet 223 is connected with a saline supply unit. The fifth outlet 222 is connected to the second outlet 1212 through the first connecting tube 3. In another words, the second connecting valve 22 does not have a venting design (i.e. anyone of the three outlets cannot be open to air). After a composite of an alternating gas-liquid formulation has been formed using the filling part 1, the four outlet 221 and the six outlet 223 can be connected to pump physiological saline into the second connecting valve 22. Then, the fourth outlet 221 can be connected to the fifth outlet 222 to push the saline into the first connecting tube 3, thereby delivering the composite of an alternating gas-liquid formulation in the second connecting tube 4 into the outlet fluid tube 6.

Similarly, the injection part 2 also includes a syringe drive part and a valve driving unit, which function in the same way respectively as the syringe drive part 13 and valve driving unit 14 in the filling part 1, so details will not be repeated here. Similarly, the injection part 2 also has a displacement sensor to detect the movement distance of the second syringe rod within the second syringe 21, and an angle sensor to detect the rotational angle of the second valve core within the second connecting valve 22.

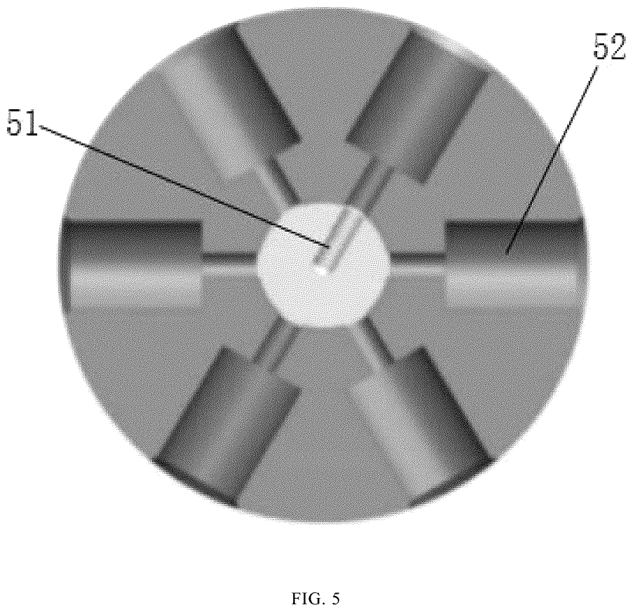

As shown in FIG. 5, in the present embodiment, the distribution valve 5 is provided with a common connection end 51 that can rotate around a center axis of the distribution valve 5. The outer wall of the distribution valve 5 is equipped with multiple distribution ports 52. In this embodiment, there are six distribution port. That is, the distribution valve 5 is a six-port valve. The number of the distribution ports 52 can be adjusted according to practical requirements. The common connection end 51 can be connected to a driving device (such as a stepper motor), allowing the common connection end 51 to rotate around the central axis of the distribution valve 5, thereby connecting to anyone of the distribution ports 52. The proximal end of the second connecting tube 4 is connected to the first connecting tube 3, and the distal end of the second connecting tube 4 is connected to the common connection port 51. One of the six distribution ports 52 is connected to the outlet fluid tube 6 (which plays a role of a distribution port for conveying liquid), while the remaining distribution ports 52 are used to connect to different gas supply parts or liquid supply parts, such as a CO₂ supply unit, a radiopaque dye supply unit, an alcohol supply unit, or a saline supply unit. By connecting the common connection end 51 to different distribution ports 52, different gases or liquids can be aspirated by the filling part 1 to provide medicinal composites with alternating gas and liquid. When the common connection end 51 is connected to the outlet fluid tube 6 through one of the distribution ports 52, medicinal composites with alternating gas and liquid can be syringed into the outlet fluid tube 6 by the injection part 2.

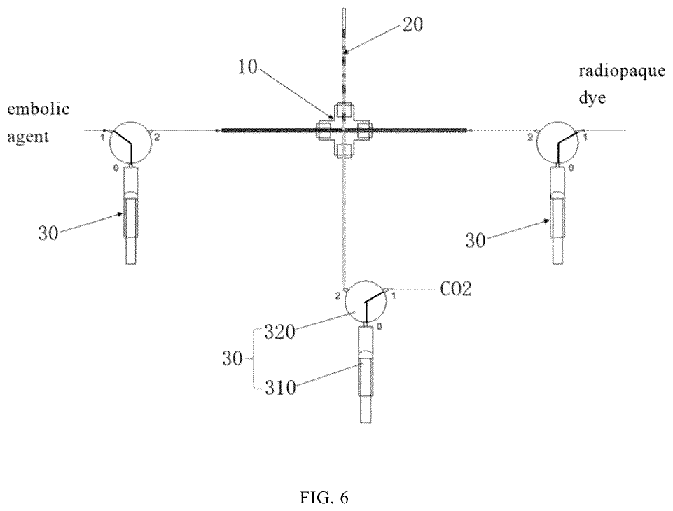

Similarly, the distribution valve 5 has multiple distribution ports (shown as FIG. 6), with one distribution port connected to the outlet fluid tube 6 (as an output port), and the remaining five distribution ports can be respectively connected to different media (gases or liquids). Thus, different media can be composited to the chain composition of alternating different media (utmost 5 kinds of media can be composited by the five distribution ports). For instance, the chain composition of alternating different media has a sequence of a first medium-a second medium-a third medium-the first medium-the second medium-the third medium-the first medium-the second medium-the third medium . . . , or the first medium-the second medium-the third medium-the second medium-the third medium-the second medium-the third medium . . . , or the first medium-the second medium . . . the fifth medium-the second medium . . . the fifth medium-the second medium . . . the fifth medium, or the first medium-the second medium-the first medium-the third medium-the first medium-the fourth medium-the first medium-the fifth medium-the first medium-the second medium-the first medium . . . etc.

It's understandable that the number of the distribution ports for the distribution valve should be at least three, but it can also be four, five, or more, depending on the need. The more distribution ports, the more kinds of media can be formulated in the chain composition.

It's important to understand that in the aforementioned example, both the filling part 1 and the injection part 2 are connected in parallel to the distribution valve 5 through the second connecting tube 4. The distribution valve 5 is located downstream of the filling part 1 and injection part 2. Specifically, during operation, the distribution valve 5 only collaborates with either the filling part 1 or the injection part 2 at any time. Moreover, the distribution valve 5 has multiple distribution ports 52, so that different distribution ports 52 are used to be in conjunction with either the filling part 1 or injection part 2.

When the distribution valve 5 is connected with the filling part 1, the common connection end 51 is adjusted to connect different distribution ports 52. At this time, the common connection end 51 is connected with the second connecting tube 4, one of the distribution ports 52 is connected to a drug supply device (for example: CO₂ supply device, saline supply device, etc.), and another distribution ports 52 is externally connected to another drug supply device (for example: CO₂ supply device, saline supply device, etc.), so as to pump gas or liquid from different drug supply devices through the distribution valve 5 and into the second connecting tube 4 by the filling part 1. Therefore, the preparation of the chain composition is completed, which is of a gas-liquid alternating chain form. The specific types of gas and liquid can be selected as needed. At this time, the injection part 2 is in a non-working state, that is, during the process of chain composition preparation, the injection part 2 is closed.

When the distribution valve 5 is connected to the injection part 2, the common connection end 51 is connected to one of the distribution ports 52 that is connected to the outlet fluid tube 6 for liquid delivery (at this point, the distribution ports 52 connected to the drug supply devices are in an idle state), thereby using the injection part 2 to push chain composition from the second connecting tube 4 into the outlet fluid tube 6 through the distribution valve 5, for the purpose of delivering the chain composition to the patient. At this time, the filling part 1 is in an idle state. That is, during the delivering process, the filling part 1 (the first connecting valve 12) is closed.

By this way, the chain composition preparation is achieved through the cooperation of the distribution valve 5 and the filling part 1, and the injection of the chain composition is achieved through the cooperation of the distribution valve 5 and the injection part 2, thereby completing the integrated operation of preparation and infusion, improving the overall convenience of the operation, and facilitating the doctor's to preparation and injection on the surgical site.

Furthermore, in the present embodiment, since the filling part 1 needs to constantly aspirate or discharge for the preparation of the chain composition, and thus is configured to have an exhaust port. However, the injection part 2 requires good sealing to prevent gas from entering the second connecting tube 4. Therefore, the injection part 2 is directly connected to an external saline supply device to directly extract physiological saline into the outlet fluid tube 6. Meanwhile, after the physiological saline has been injected, the second connecting tube 4 remains filled with physiological saline, without any gas.

As shown in FIG. 1, in the present embodiment, the chain composition preparation apparatus further includes a pressure sensor 7, which is installed on the first connecting tube 3 between the fifth valve outlet 222 and the proximal end of the second connecting tube 4. This allows the pressure sensor 7 to detect the pressure values within the first connecting tube 3 and the second connecting tube 4, ensuring that the pressure within the chain composition preparation apparatus remains within the normal range.

In the present embodiment, both the first connecting tube 3 and the second connecting tube 4 are made of Teflon. However, there's no limitation to the specific material of the first connecting tube 3 and the second connecting tube 4. In other instances, connecting tubes made of other medical materials can also be selected.

### Second Embodiment

Based on the chain composition preparation apparatus in the first embodiment, the present embodiment provides an injection method for chain composition, which specifically includes the following steps:

### S1: Using the Filling Part 1 for Exhausting the Air.

Before injection, completely exhausting the air from the chain composition preparation apparatus to prevent the air from being injected into the human body, which could potentially cause harm. The step of S1 include S11 to S15:

S11: Connecting the first valve outlet 1211 to the second valve outlet 1212, and connecting the fourth valve outlet 221 to the fifth valve outlet 222. At this time, the head of the first syringe rod 112 is located at the distal end of the first syringe tube 111, and the head of the second syringe rod is located at the distal end of the second syringe tube. Meanwhile, the common connection end 51 is driven to connect to the saline supply device via the distribution port 52.

S12: Moving the first syringe rod 112 by the syringe drive unit 13, thereby aspirating the air from the first connecting tube 3 and the second connecting tube 4. Simultaneously, it aspirates in physiological saline into the second connecting tube 4. Each suction of the air has a volume equivalent to the capacity of the first syringe tube 111. In this embodiment, a maximum of 250 μl of air can be aspirated each time.

S13: Controlling the rotation of the first valve core 122 through the valve drive part 14, ensuring that the first valve outlet 1211 connects to the third valve outlet 1213.

S14: moving the first syringe rod 112 by the syringe drive unit 13, allowing the air to be expelled through the third valve outlet 1213. Subsequently, the first syringe rod 112 returns to an initial position as in step S11, ready for aspirating the air next time.

S15: Controlling the rotation of the first valve core 122 by the valve drive part 14, ensuring that the first valve outlet 1211 connects to the second valve outlet 1212.

S16: Repeating steps S11 to S15 until the first connecting tube 3 and the second connecting tube 4 are filled with physiological saline.

### S2: Preparing the Chain Composition Using the Filling Part 1.

Once the air is exhausted from both the first connecting tube 3 and the second connecting tube 4, different chain composition are prepared according to the patient's needs. Taking the preparation of a gas-liquid alternating chain composition of 10 μl CO₂+10 μl alcohol as an example, the specific steps include:

S21: Controlling the rotation of the first valve core 122 through the valve driving unit 14, ensuring communication between the first valve outlet 1211 and the second valve outlet 1212. Additionally, using the drive device to connect the common connection end 51 to the CO₂ supply device through the distribution port 52.

S22: Moving the first syringe rod 112 by the syringe drive unit 13 to suck 10 μl of saline from the first connecting tube 3 and the second connecting tube 4. Simultaneously, 10 μl of CO₂ is sucked into the second connecting tube 4.

S23: Controlling the rotation of the first valve core 122 by the valve driving unit 14, ensuring communication between the first valve outlet 1211 and the third valve outlet 1213.

S24: Moving the first syringe rod 112 by the syringe drive part 13, allowing 10 μl of CO₂ from the first syringe tube 111 to be expelled outside through the third valve outlet 1213.

S25: Controlling the rotation of the first valve core 122 by the valve drive unit 14, connecting the first valve outlet 1211 to the second valve outlet 1212, and connecting the common connection end 51 to the distribution port 52 that is connected to an alcohol supply device.

S26: Moving the first syringe rod 112 by the syringe drive unit 13, thereby aspirating 10 μl of saline from the first connecting tube 3 and the second connecting tube 4. Simultaneously, 10 μl of alcohol is aspirated into the second connecting tube 4.

S27: Controlling the rotation of the first valve core 122 by the valve driving unit 14, connecting the first valve outlet 1211 to the third valve outlet 1213.

S28: Moving the first syringe rod 112 by the syringe drive part 13, allowing 10 μl of fluid from the first syringe tube 111 to be exhausted from the third valve outlet 1213.

S29: Controlling the rotation of the first valve core 122 through the valve driving unit 14, connecting the first valve outlet 1211 to the second valve outlet 1213. Repeat steps S21-S28 until a set volume of gas-liquid alternating agent (chain composition) is prepared within the second connecting tube 4.

It is understandable that in the present embodiment, the gas-liquid alternating agent formulation with 10 μl CO₂ and 10 μl alcohol is only one implementation. In other embodiments, formulations with three or four materials can also be implemented. The specific method is similar to the above steps, just by connecting the common connection end 51 with the distribution ports 52 to different agent containers, which will not be elaborated here again.

S3: Using the Injection Part 2 to Deliver the Chain Composition into the Outlet Fluid Tube 6.

Once the chain composition is prepared, it needs to be injected into the patient. This process is carried out by the injection part 2, while the filling part 1 remains in standby mode. The process involves the following steps:

S31: Controlling the rotation of the second valve core by the valve driving unit of the injection part 2, allowing the fourth valve outlet 221 to connect the sixth valve outlet 223. And connecting the common connection end 51 to the outlet fluid tube 6 through the distribution port 52.

S32: Driving the second syringe rod by the syringe drive unit of the injection part 2 to aspirate a specific volume of saline into the second syringe through the sixth valve outlet 223. This specific volume of saline is determined based on the volume of the chain composition needed.

S33: Controlling the rotation of the second valve core by the push unit 2, allowing the fourth valve outlet 221 to connect the fifth valve outlet 222.

S34: Driving the second syringe rod by the injection part 2 to inject a specific volume of saline from the injection part 2 into the first connecting tube 3. Since the first connecting tube 3 is filled with saline and the second connecting tube 4 is already filled with prepared chain composition, the saline introduced into the first connecting tube 3 from the injection part 2 pushes the chain composition, consisting of 10 μl CO₂ and 10 μl alcohol, from the distal end of the second connecting tube 4, through the distribution valve 5, and into the outlet fluid tube 6.

It's important to understand that since the chain composition at the distal end of the second connecting tube 4 has already been pushed into the outlet fluid tube 6, the second connecting tube 4 is now refilled with saline from the first connecting tube 3. This allows for the next preparation of the chain composition by the aforementioned steps.

### S4: Connecting the Outlet Fluid Tube 6 to an Injection Unit for Injecting the Chain Composition.

In summary, the chain composition preparing apparatus and preparing method provided in the embodiments of the invention can integrate the preparation of the chain composition and its injection, thereby enhancing the convenience of injection and injection efficiency. Furthermore, by selecting a distribution valve with three or more distribution ports, it is possible to prepare a chain composition of alternating different media using only one filling part and one injection part. Especially when achieving a chain composition of alternating more than 3 different media, it only requires one filling part and one injection part, thereby improving preparation efficiency, reducing the size of the chain composition preparation apparatus, and lowering manufacturing costs.

### Third Embodiment

As shown in FIG. 6, the third embodiment of this invention provides a chain composition preparation apparatus that includes a distribution valve 10, an outlet fluid tube 20, and multiple filling parts 30. The distribution valve 10 connects the outlet fluid tube 20 with the multiple filling parts 30, allowing different media to be pushed into the outlet fluid tube 20 through these multiple filling parts 30, thereby forming a chain composition with alternating media.

Specifically, in the present embodiment, the distribution valve 10 is a four-way valve, having four interconnected distribution ports. One of these ports is used to connect with the outlet fluid tube 20, while the other three ports are respectively connected to three filling parts 30.

In the present embodiment, each of the filling parts 30 includes a first syringe 310 and a first connecting valve 320. The first connecting valve 320 at least has three ports: a first valve outlet, a second valve outlet, and a third valve outlet. The first syringe 310 is connected to the first valve outlet. The second valve outlet is connected to the distribution valve 10 through one of the distribution ports. The third valve outlet is used to connect with the gas supply part or the liquid supply part. Specifically, in this embodiment, the three filling parts 30 are respectively connected to the CO₂ supply unit, the radiopaque dye (a contrast agent) supply unit, and the embolic agent supply unit. Each of the filling parts 30 can reciprocate, drawing gas or liquid from the gas supply part or liquid supply part and delivering it into the outlet fluid tube 20. It also draws some gas from the outlet fluid tube 20 to isolate some fluid from the rest of the liquid, thereby forming a chain-like composition with alternating media as described above.

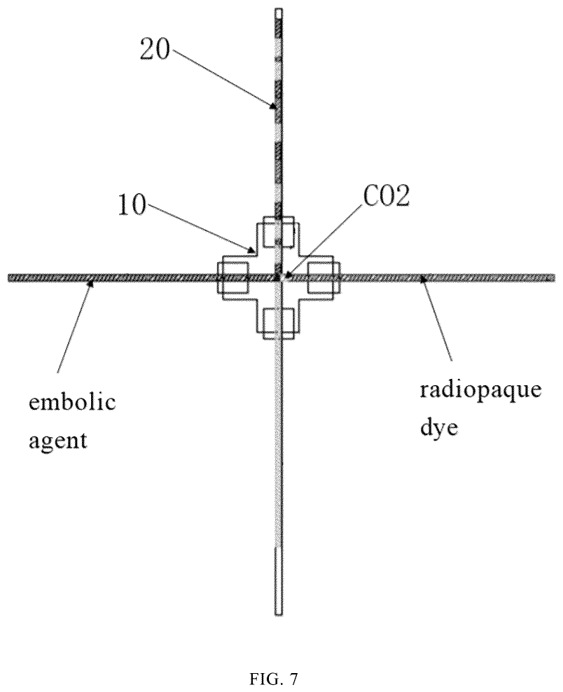

The following provides a detailed method for preparing a chain composition with alternating media:
Step 1: aspirating the first medium (such as a contrast agent) from the liquid supply part through the first filling part 30, and push it into the outlet fluid tube 20 through the distribution valve 10;
Step 2: aspirating gas (such as CO₂) from the gas supply device through the second filling part 30, and pushing it into the outlet fluid tube 20 through the distribution valve 10;
Step 3: aspirating a preset volume gas from the outlet fluid tube 20 through the first filling part 30 to isolate the first medium from the rest of the medium (as shown in FIG. 7);
Step 4: aspirating the second medium (e.g., embolic agent) from another liquid supply part through the third filling part, and then pushing it into the outlet fluid tube 20 through the distribution valve 10;
Step 5: aspirating gas from the gas supply device through the second filling part and push it into the outlet fluid tube 20 through the distribution valve;
Step 6: aspirating a preset volume of gas from the outlet fluid tube 20 through the third filling part, to isolate the second medium from the rest of the medium;
Step 7: aspirating the Nth medium from another liquid supply part through the Nth filling part, and pushing it into the outlet fluid tube 20 through the distribution valve. This process is repeated sequentially, until a chain-like composition of alternating media is prepared.

The Nth medium is different from the neighbouring medium within the outlet fluid tube.

It is understandable that the alternating chain composition of different media formed in this way includes at least one gas medium and two liquid media. The number of liquid media can be adaptively increased or decreased as needed.

The above provides a detailed explanation of this invention. For those skilled in the art, any apparent alterations made to it without departing from the essential contents will constitute a violation of the patent rights of this invention, and they will bear the corresponding legal responsibilities.

What is claimed is:

1. A chain composition preparation apparatus, comprising:
a distribution valve, comprising a plurality of distribution ports, which are capable to interconnect with a common connecting end;
a filling part, connected to one of the distribution ports through a first connecting tube, allowing gas or liquid to enter the first connecting tube from the distribution valve, to prepare a chain composition of alternated media;
an injection part, connected to another one of the distribution ports through the first connecting tube, for delivering the chain composition within the first connecting tube;
an outlet fluid tube, connected to other one of the distribution ports to deliver the chain composition from the distribution valve, and
a second connecting tube, connecting the common connecting end to the first connecting tube,
the first connecting tube connecting the filling part, the injection part and the second connecting tube at different ends.

2. The chain composition preparation apparatus as claimed in claim 1, wherein
the filling part comprises a first syringe and a first connecting valve,
the first connecting valve at least has a first valve outlet, a second valve outlet and a third valve outlet, which are one-to-one connectable,
the first syringe is connected to the first valve outlet, the first connecting tube is connected to the second valve outlet, and the third valve outlet is configured to exhaust.

3. The chain composition preparation apparatus as claimed in claim 2, wherein
the injection section comprises a second syringe and a second connecting valve,
the second connecting valve at least has a fourth valve outlet, a fifth valve outlet, and a sixth valve outlet, which are one-to-one connectable,
the second syringe is connected to the fourth valve outlet,
the fifth valve outlet is connected to the second valve outlet through the first connecting tube,
the sixth valve outlet is configured to aspirate liquid.

4. The chain composition preparation apparatus as claimed in claim 3, wherein
the distribution valve further has a plurality of distribution ports in an outer wall thereof,
the common connecting end is rotatable around a central axis of the distribution valve to communicate with any one of the distribution ports;
the second connecting tube is connected to the first connecting tube at a proximal end, while the second connecting tube is connected to the common connecting port at a distal end,
one of the distribution ports is connected to the outlet fluid tube, while the remaining distribution ports is capable to connect to different gas supply parts or liquid supply parts.

5. The chain composition preparation apparatus as claimed in claim 3, wherein
the chain composition preparation apparatus further comprises a pressure sensor, wherein the pressure sensor is installed on the first connecting tube between the fifth valve outlet and a proximal end of the second connecting tube.

6. (canceled)

7. The chain composition preparation apparatus as claimed in claim 2, wherein
the first syringe comprises a first syringe tube and a first syringe rod,
the first syringe tube is connected to the first valve outlet at a distal end thereof, and the first syringe rod is movably inserted inside the first syringe tube,
the first syringe rod is driven to reciprocate inside the first syringe tube.

8. The chain composition preparation apparatus as claimed in claim 2, wherein
the filling part further comprises a valve driving unit, and the first connecting valve comprises a first valve body and a first valve core,
the first valve body has a central hole extending axially, and an outer wall of the first valve body is provided with the first valve outlet, the second valve outlet, and the third valve outlet, all of which extend radially and are connected to the central hole;
the first valve core has a curved passage that runs through its outer wall,
the first valve core is rotatably placed inside the central hole and is connected to the valve driving unit,
the first valve core is capable to rotate around the central axis of the first valve body, allowing both ends of the curved passage to communicate with any two of the first valve outlet, the second valve outlet, and the third valve outlet.

9. A chain composition preparation method using the chain composition preparation apparatus as claimed in claim 1, comprising the following steps:
using the filling part for exhausting the air until the first connecting tube and the second connecting tube are filled with physiological saline;
preparing the chain composition using the filling part, by aspirating gas from a gas supply device and aspirating liquid from a liquid supply device alternatively into the second connecting tube, so as to form a chain-like composition of alternating media;
using the injection part to deliver the chain composition into the outlet fluid tube.

10. The method as claimed in claim 9, wherein including
using the distribution valve to formulate a chain composition with alternating different media,
the chain composition comprises at least two different media, which are alternatively lined in a chain-like form.

11. A chain composition preparation apparatus, comprising:
a distribution valve, comprising a plurality of interconnected distribution ports;
an outlet fluid tube, connected to one of the distribution ports of the distribution valve; and
a plurality of filling parts, each connected to one of distribution port of the distribution valve other from the one connected to the outlet fluid tube, and each connected to a gas supply part or a liquid supply part, so as to allow gases or liquids to flow through the distribution valve into the outlet fluid tube to prepare a chain composition with different media alternating.

12. The chain composition preparation apparatus as claimed in claim 11, wherein
the filling parts are configured to reciprocate and aspirate gas or liquid from the gas supply parts or liquid supply parts and deliver the gas or liquid into the outlet fluid tube,
the filling parts are further configured to aspirate gas from the outlet fluid tube to isolate the liquid from the rest of the liquid.

13. The chain composition preparation apparatus as claimed in claim 11, wherein
the filling part comprises a first syringe and a first connecting valve,
the first connecting valve at least has a first valve outlet, a second valve outlet and a third valve outlet, which are connectable with each other,
the first syringe being connected to the first valve outlet,
the second valve outlet is connected to the distribution valve through one of its distribution ports,
the third valve outlet is configured to connect with the gas supply part or the liquid supply part.

14. The chain composition preparation apparatus as claimed in claim 11, wherein
among the plurality of filling parts, only one of them is configured to connect with the gas supply part, while the rest are used to connect with different liquid supply parts.

15. A chain composition preparation method using the chain composition preparation apparatus as claimed in claim 11, comprising the following steps:
aspirating a first medium from a liquid supply part through a first filling part, and push it into the outlet fluid tube through the distribution valve,
aspirating gas from a gas supply device through a second filling part, and pushing it into the outlet fluid tube through the distribution valve,
aspirating gas from the outlet fluid tube through the first filling part to isolate the first medium from the rest of the medium,
aspirating a second medium from another liquid supply part through a third filling part, and then pushing it into the outlet fluid tube through the distribution valve;
aspirating gas from the gas supply device through the second filling part and push it into the outlet fluid tube through the distribution valve;
aspirating gas from the outlet fluid tube through the third filling part, to isolate the second medium from the rest of the medium;
aspirating a Nth medium from a Nth liquid supply part through a Nth filling part, and pushing it into the outlet fluid tube through the distribution valve,
repeating above steps until a chain composition of alternating media is prepared,
wherein, the Nth medium is different from the first medium or the second medium within the outlet fluid tube.

16. A chain composition preparation method using the chain composition preparation apparatus as claimed in claim 2, comprising the following steps:
using the filling part for exhausting the air until the first connecting tube and the second connecting tube are filled with physiological saline;
preparing the chain composition using the filling part, including the following steps:
connecting the first valve outlet and the second valve outlet, and connecting the common connection end to a gas supply device through the distribution port;
moving the first syringe rod to aspirate the physiological saline from the first connecting tube and the second connecting tube until gas is aspirated into the second connecting tube;
connecting the first valve outlet and the third valve outlet;
moving the first syringe rod to allow the gas from the first syringe tube to be expelled outside through the third valve outlet;
connecting the first valve outlet to the second valve outlet, and connecting the common connection end to a distribution port being configured to connect with a liquid supply device;
moving the first syringe rod to aspirating the physiological saline from the first connecting tube and the second connecting tube until fluid is aspirated into the second connecting tube;
connecting the first valve outlet to the third valve outlet;
moving the first syringe rod to allow the fluid from the first syringe tube to be exhausted from the third valve outlet;
connecting the first valve outlet to the second valve outlet;
repeat the aforementioned steps until the chain composition is prepared within the second connecting tube;
using the injection part to deliver the chain composition into the outlet fluid tube.
